(19) United States
(12) Patent Application Publication
(10) Pub. No.: US 2020/0031748 A1
(43) Pub. Date: Jan. 30, 2020

(54) Mixture for Use as a Liquid Sorption Agent in Methanol Synthesis and Methanol Synthesis Process Using Said Mixture

(71) Applicant: Siemens Aktiengesellschaft, München (DE)

(72) Inventors: Manfred Baldauf, Erlangen (DE); Alexander Tremel, Möhrendorf (DE); Jakob Albert, Erlangen (DE); Katharina Meltzer, Erlangen (DE); Jenny Reichert, Schwanfeld (DE); Peter Wasserscheid, Erlangen (DE)

(73) Assignee: Siemens Aktiengesellschaft, München (DE)

(21) Appl. No.: 16/493,989

(22) PCT Filed: Mar. 12, 2018
(86) PCT No.: PCT/EP2018/055997

§ 371 Date: Sep. 13, 2019

(30) Foreign Application Priority Data
Mar. 14, 2017 (DE) 10 2017 204 226.5

(51) Int. Cl. C07C 29/94; C07C 317/28

(52) U.S. Cl. CPC C07C 29/94; C07C 317/28

(57) ABSTRACT

Some embodiments include a mixture for use as liquid sorbent for methanol or methanol and water in methanol synthesis using carbon monoxide and hydrogen, carbon dioxide and hydrogen or a mixture of hydrogen, carbon monoxide and carbon dioxide as synthesis reactants. The mixture comprises I) a component A) in the form of at least one salt and at least one component B) selected from the group consisting of: B1) a salt of an anion and one, two, or three of the cations of salt A), wherein the number of cations corresponds to an absolute value of a charge number of the respective anion; B2) a salt comprising one or more bis(trifluoromethylsulfonyl)imide anions and another component, wherein a number of bis(trifluoromethylsulfonyl)imide anions corresponds to an absolute value of a charge number of the respective metal cation; and B3) a zwitterionic compound.

### CROSS-REFERENCE TO RELATED APPLICATIONS

This application is a U.S. National Stage Application of International Application No. PCT/EP2018/055997 filed Mar. 12, 2018, which designates the United States of America, and claims priority to DE Application No. 10 2017 204 226.5 filed Mar. 14, 2017, the contents of which are hereby incorporated by reference in their entirety.

### TECHNICAL FIELD

The present disclosure relates to methanol synthesis. Various embodiments include mixtures for use as liquid sorbent in methanol synthesis and/or methods for conducting a methanol synthesis using the mixture.

### BACKGROUND

In many chemical syntheses, what are known as equilibrium-limited reactions occur, that is to say the synthesis of products stops (on the face of it) when an equilibrium position between reactants and product(s) has been reached. In the case of such syntheses, for a high degree of conversion it is accordingly advantageous to remove product(s) from the synthesis system in order thus to avoid the establishment of an equilibrium position between reactants and product(s) or in order to come back to a disequilibrium position from an equilibrium position, which for example can be achieved by a continuous or discontinuous removal of product(s) from the reaction system.

Two known examples of a chemical synthesis subject to equilibrium-limited reaction are the production of methanol from hydrogen and carbon monoxide as per reaction equation 1 below or from hydrogen and carbon dioxide as per reaction equation 2 below:

2H₂+CO [symbol] CH₃OH 1.

3H₂+CO₂ [symbol] CH₃OH+H₂O 2.

The synthesis of methanol currently takes place primarily in heterogeneous, catalyzed fixed-bed reactors, where the reactants are only partially converted during a single pass through the reactor. The condensable reaction products are thus separated off after one pass and the unconverted reactants recirculated to the reaction inlet. Recirculation of the, sometimes large, amounts of gas leads to high apparatus complexity.

In order to overcome or else lessen this disadvantage, DE 10 2015 202 681 A1 discloses a method for carrying out a chemical synthesis using two carrier phases, which are soluble in one another only to some extent, and a catalyst, which is more dispersed in one of the carrier phases, in which the carrier phases are mixed in a reactor, at least one synthesis reactant is introduced into the reactor and the two carrier phases are subsequently separated from one another.

In methanol synthesis, the synthesis product methanol/the synthesis products methanol and water have an affinity for polar liquids, and so the synthesis product/the synthesis products accumulates in such liquids. Therefore, according to the mentioned publication, one of the carrier phases in the methanol synthesis is preferably a polar liquid, which may be formed from ionic liquid(s), polar solvent(s) or else one or more higher molecular weight alcohols.

The synthesis product(s) accumulate(s) in the polar carrier phase, the result being that, until the saturation limit is reached in the polar carrier phase, the establishment of a steady-state equilibrium situation is prevented in the other, less polar or non-polar carrier phase that includes (at least predominantly) the reactants and the catalyst which is required for the methanol synthesis.

In a further method step, the two carrier phases are separated from one another by, for example, the polar carrier phase being guided out of the reactor, and the synthesis product(s) (methanol or methanol and water) are at least partially separated off from the polar carrier phase. The polar carrier phase “freed” of the synthesis products can thereafter be recycled back into the reactor.

The ionic liquids mentioned as possible carrier phase are salts which melt at low temperatures (<100° C.) and constitute a class of solvents having an extremely low vapor pressure.

Even though initial representatives had been known since as early as 1914, it is only in the last approximately 15 years that ionic liquids have been investigated intensively as solvents for chemical reactions. The extremely low vapor pressure of ionic liquids is of great advantage for certain methods, since an extractive separation of a reaction mixture is thus possible without contaminating the gas phase with solvent vapors. The recovery of the extract from the ionic liquid is often possible by simple distillation, where problems such as formation of an azeotrope between solvent and extract do not arise.

Suitable choice of cation and anion of an ionic liquid enables a specific adjustment of the polarity thereof and hence a tuning of the solubility properties thereof. In this case the spectrum extends from water-miscible ionic liquids, through water-immiscible ionic liquids, up to those that even form two phases with organic solvents or other ionic liquids. Skillful exploitation of the exceptional solubility properties is the key to successful use of ionic liquids as a novel class of solvents.

Problems with the use of ionic liquids can arise at relatively high temperatures since many of the ionic liquids are not permanently temperature-stable above 200° C. The presence of catalysts, reactants and reaction products can also result in a significant narrowing of the choice of cation and anion from considerations of stability.

P. Wasserscheid, W. Keim: “Ionische Flüssigkeiten—neue “Lösungen” für die Übergangsmetallkatalyse” [Ionic liquids—novel “solutions” for transition metal catalysis], Angew. Chem., volume 112, edition 21, pp. 3926-3945, 3 Nov. 2000, describe the use of ionic liquids as extraction media in two-phase catalysis.

Even though ionic liquids have already been mentioned in principle in the prior art as possible carrier phase in the context of methanol synthesis, a problem that has not been solved to date is the use of ionic liquids as sorbent (extraction phase) of methanol or methanol and water under the reaction conditions of methanol gas-phase synthesis using carbon monoxide and hydrogen, carbon dioxide and hydrogen or a mixture of carbon monoxide, carbon dioxide and hydrogen. This is because, in this case, there are particularly high demands on the stability of the ionic liquid(s) used at up to 300° C. and up to 300 bar synthesis gas pressure in the presence of a catalyst, such as for example Cu—Zn oxide on aluminum oxide, and of the reaction products.

It would also be advantageous if, in the case of a methanol synthesis using carbon dioxide and hydrogen, the ionic liquid were also to have a sorption performance as equally good as possible in respect of both of the synthesis products water and methanol in order to avoid an accumulation of one of the synthesis products in the gas phase.

### SUMMARY

Against this background, the teachings of the present disclosure describe novel sorbents for methanol or methanol and water in methanol synthesis based on ionic liquids and also a method for methanol synthesis using sorbents of this kind. For example, some embodiments include a mixture for use as liquid sorbent for methanol or methanol and water in methanol synthesis using carbon monoxide and hydrogen, carbon dioxide and hydrogen or a mixture of hydrogen, carbon monoxide and carbon dioxide as synthesis reactants, characterized in that the mixture consists of
I) a component A) in the form of at least one salt, which is formed from the
bis(trifluoromethylsulfonyl)imide anion

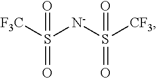 [SMILES: O=S(=O)([N-]S(=O)(=O)C(F)(F)F)C(F)(F)F]

and a cation in the form of a
   quaternary ammonium cation of the general formula [NR¹R²R³R]⁺; or
   a phosphonium cation of the general formula [PR¹R²R³R]⁺; or
   an imidazolium cation of the general formula

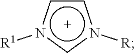

or
a pyridinium cation of the general formula

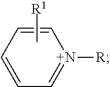

or
a pyrazolium cation of the general formula

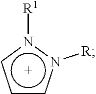

or
a triazolium cation of the general formula

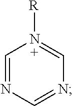

wherein, on the imidazole ring, pyridine ring, pyrazole ring or triazole ring, one or more hydrogen radicals may be substituted by a group which is independently selectable from C₁-C₆-alkyl, C₁-C₆-alkoxy, C₁-C₆-aminoalkyl, C₅-C₁₂-aryl and C₅-C₁₂-aryl-C₁-C₆-alkyl groups, wherein the alkyl, alkoxy and aminoalkyl groups may be linear or branched, saturated or unsaturated, aliphatic or alicyclic and the aryl radical can have 6 to 20 carbon atoms;
the radicals R¹, R², R³ are independently selected from hydrogen; linear or branched, saturated or unsaturated, aliphatic or alicyclic alkyl groups having 1 to 20 carbon atoms, where up to 6 hydrogen radicals may be substituted by OH groups; heteroaryl groups and/or heteroaryl-C₁-C₆-alkyl groups having 3 to 8 carbon atoms in the heteroaryl radical and at least one heteroatom in the heteroaryl radical that is selected from N, O and S, wherein the alkyl groups may be linear or branched, saturated or unsaturated, aliphatic or alicyclic and wherein one or more hydrogen radicals on the heteroaryl radical may be substituted by at least one group which is independently selectable from linear or branched, saturated or unsaturated, aliphatic or alicyclic C₁-C₆-alkyl groups and/or halogen atoms; aryl and/or aryl-C₁-C₆-alkyl groups having 6 to 20 carbon atoms in the aryl radical, wherein the alkyl groups may be linear or branched, saturated or unsaturated, aliphatic or alicyclic and wherein on the aryl radical one or more hydrogen radicals may be substituted by at least one group which is independently selectable from linear or branched, saturated or unsaturated, aliphatic or alicyclic C₁-C₆-alkyl groups and/or halogen atoms; and —[—(CH₂)ₓ—O—]_{y}—CH₃ groups where x=2-5 and y=1-20; and
the radical R is selected from linear or branched, saturated or unsaturated, aliphatic or alicyclic alkyl groups having 1 to 20 carbon atoms, where up to 6 hydrogen radicals may be substituted by OH groups; heteroaryl-C₁-C₆-alkyl groups having 3 to 8 carbon atoms in the heteroaryl radical and at least one heteroatom in the heteroaryl radical that is selected from N, O and S, wherein the alkyl groups may be linear or branched, saturated or unsaturated, aliphatic or alicyclic and wherein one or more hydrogen radicals on the heteroaryl radical may be substituted by at least one group which is independently selectable from linear or branched, saturated or unsaturated, aliphatic or alicyclic C₁-C₆-alkyl groups and/or halogen atoms; aryl-C₁-C₆-alkyl groups having 6 to 20 carbon atoms in the aryl radical, wherein the alkyl groups may be linear or branched, saturated or unsaturated, aliphatic or alicyclic and wherein one or more hydrogen radicals on the aryl radical may be substituted by at least one group which is independently selectable from linear or branched, saturated or unsaturated, aliphatic or alicyclic C₁-C₆-alkyl groups and/or halogen atoms; and —[—(CH₂)ₓ—O—]_{y}—CH₃ groups where x=2-5 and y=1-20;and
II) a component B) which consists of at least one of the following components:
B1) a salt that is formed from one of the anions [PO₄]³⁻, [HPO₄]²⁻, [H₂PO₄]⁻, [SO₄]²⁻, [HSO₄]⁻, [NO₃]⁻, [NO₂]⁻ or Cl⁻ and one, two or three of the cations stated in A), wherein the number of cations corresponds to the absolute value of the charge number of the respective anion;
B2) a salt that is formed from one or more
bis(trifluoromethylsulfonyl)imide anions

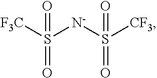 [SMILES: O=S(=O)([N-]S(=O)(=O)C(F)(F)F)C(F)(F)F]

and a lithium, potassium, cesium, magnesium, calcium, barium, nickel, cobalt, iron, scandium, lanthanum, zinc, gallium, cerium or aluminum cation, wherein the number of bis(trifluoromethylsulfonyl)imide anions corresponds to the absolute value of the charge number of the respective metal cation; and/or
B3) a zwitterionic compound that is formed from one of the cations stated in A), in which one of the radicals R, R², R² or R³ is a —(CH₂)ₓ—SO₃⁻ group where x=1-10.

In some embodiments, the proportion by mass of component B) in the mixture is in the range from 1% to 99%, e.g. in the range from 1% to 80%.

In some embodiments, it is liquid at a temperature from 79° C., or from 49° C., or from 19° C.

In some embodiments, it consists of:
a) tributylmethylphosphonium bis(trifluoromethylsulfonyl)imide and lithium bis(trifluoromethylsulfonyl)imide;
b) methyltrioctylphosphonium bis(trifluoromethylsulfonyl)imide and lithium bis(trifluoromethylsulfonyl)imide;
c) trihexyltetradecylphosphonium bis(trifluoromethylsulfonyl)imide and lithium bis(trifluoromethylsulfonyl)imide;
d) tributylmethylphosphonium bis(trifluoromethylsulfonyl)imide and cesium bis(trifluoromethylsulfonyl)imide;
e) tributylmethylphosphonium bis(trifluoromethylsulfonyl)imide, lithium bis(trifluoromethylsulfonyl)imide and tributyl-4-sulfonyl-1-butanephosphonium;
f1) tributylmethylphosphonium bis(trifluoromethylsulfonyl)imide and tris(tetrabutylphosphonium) phosphate;
f2) tributylmethylphosphonium bis(trifluoromethylsulfonyl)imide, tris(tetrabutylphosphonium) phosphate and lithium bis(trifluoromethylsulfonyl)imide;
g1) tributylmethylphosphonium bis(trifluoromethylsulfonyl)imide and bis(tetrabutylphosphonium) sulfate;
g2) tributylmethylphosphonium bis(trifluoromethylsulfonyl)imide, bis(tetrabutylphosphonium) sulfate and lithium bis(trifluoromethylsulfonyl)imide;
h) tributylmethylphosphonium bis(trifluoromethylsulfonyl)imide, tetrakis(hydroxymethyl)phosphonium bis(trifluoromethylsulfonyl)imide and lithium bis(trifluoromethylsulfonyl)imide;
i) tributylmethylphosphonium bis(trifluoromethylsulfonyl)imide, tributylhydroxymethylphosphonium bis(trifluoromethylsulfonyl)imide and lithium bis(trifluoromethylsulfonyl)imide; or
j) tributylmethylphosphonium bis(trifluoromethylsulfonyl)imide, tributyl-2,3-dihydroxypropylphosphonium bis(trifluoromethylsulfonyl)imide and lithium bis(trifluoromethylsulfonyl)imide.

In some embodiments, for binary mixtures, the mass ratio of the first component specified to the second component specified is in the range from 4:1 to 7:3; and for ternary mixtures, the mass ratio of the first component specified to the second component specified to the third component specified is in the range from 4:1:1 through 4:3:1 to 4:3:3.

As another example, some embodiments include a method for conducting a methanol synthesis using carbon monoxide and hydrogen, carbon dioxide and hydrogen or a mixture of carbon monoxide, carbon dioxide and hydrogen as synthesis reactants in a reactor at a temperature in the range from 100° C. to 300° C. and a synthesis gas pressure in the range from 50 bar to 300 bar in the presence of a catalyst, comprising the following steps: providing a liquid mixture as described above in the reactor; converting carbon monoxide and hydrogen, carbon dioxide and hydrogen or a mixture of carbon monoxide, carbon dioxide and hydrogen to the synthesis product methanol or the synthesis products methanol and water in the reactor at a temperature in the range from 100° C. to 300° C. and a synthesis gas pressure in the range from 50 bar to 300 bar; sorbing at least one subamount of at least one synthesis product, from the gas phase comprising at least one synthesis product, into the liquid mixture; and continuously or discontinuously guiding the liquid mixture out of the reactor.

In some embodiments, the gas phase comprising at least one synthesis product is introduced into the mixture by means of a sparging stirrer.

In some embodiments, the liquid mixture guided out of the reactor is depressurized and in that at least the methanol—at least partially emerging from the mixture as a result—is recovered.

In some embodiments, after the depressurization of the liquid mixture and the at least partial emergence from the mixture of the synthesis product methanol or the synthesis products methanol and water, the mixture is recycled back into the reactor.

In some embodiments, the catalyst used is copper/zinc oxide on aluminum oxide, said catalyst being arranged in the synthesis-gas gas phase of the reactor.

In some embodiments, the synthesis gas used is carbon dioxide and hydrogen; at least subamounts of the synthesis products methanol and water are absorbed into the liquid mixture in the reactor; the guidance of the mixture out of the reactor and the depressurization of the mixture is followed by the synthesis products methanol and water at least partially emerging from the mixture; at least the synthesis product methanol that has at least partially emerged is recovered; and the depressurization of the liquid mixture and the at least partial emergence of the synthesis products from the mixture is followed by the mixture being recycled back into the reactor.

In some embodiments, heat is extracted from the mixture prior to recycling into the reactor.

### DETAILED DESCRIPTION

The teachings of the present disclosure describe mixtures for use as liquid sorbent for methanol or methanol and water in methanol synthesis using carbon monoxide and hydrogen, carbon dioxide and hydrogen or a mixture of carbon monoxide, carbon dioxide and hydrogen as synthesis reactants. In some embodiments, the mixture is characterized in that it consists of I) a component A) in the form of at least one salt, which is formed from the bis(trifluoromethylsulfonyl)imide anion

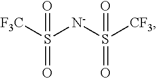 [SMILES: O=S(=O)([N-]S(=O)(=O)C(F)(F)F)C(F)(F)F]

and a cation in the form of a
quaternary ammonium cation of the general formula [NR¹R²R³R]⁺; or
a phosphonium cation of the general formula [PR¹R²R³R]⁻; or
an imidazolium cation of the general formula

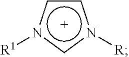

or
a pyridinium cation of the general formula

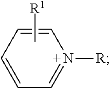

or
a pyrazolium cation of the general formula

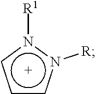

or
a triazolium cation of the general formula

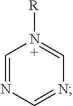

wherein, on the imidazole ring, pyridine ring, pyrazole ring or triazole ring, one or more hydrogen radicals may be substituted by a group which is independently selectable from C₁-C₆-alkyl, C₁-C₆-alkoxy, C₁-C₆-aminoalkyl, C₅-C₁₂-aryl and C₅-C₁₂-aryl-C₁-C₆-alkyl groups, wherein the alkyl, alkoxy and aminoalkyl groups may be linear or branched, saturated or unsaturated, aliphatic or alicyclic and the aryl radical can have 6 to 20 carbon atoms;
the radicals R¹, R², R³ are independently selected from hydrogen; linear or branched, saturated or unsaturated, aliphatic or alicyclic alkyl groups having 1 to 20 carbon atoms, where up to 6 hydrogen radicals may be substituted by OH groups; heteroaryl groups and/or heteroaryl-C₁-C₆-alkyl groups having 3 to 8 carbon atoms, e.g. 4 to 8 carbon atoms, in the heteroaryl radical and at least one heteroatom in the heteroaryl radical that is selected from N, O and S, wherein the alkyl groups may be linear or branched, saturated or unsaturated, aliphatic or alicyclic and wherein one or more hydrogen radicals on the heteroaryl radical may be substituted by at least one group which is independently selectable from linear or branched, saturated or unsaturated, aliphatic or alicyclic C₁-C₆-alkyl groups and/or halogen atoms; aryl and/or aryl-C₁-C₆-alkyl groups having 6 to 20 carbon atoms in the aryl radical, wherein the alkyl groups may be linear or branched, saturated or unsaturated, aliphatic or alicyclic and wherein on the aryl radical one or more hydrogen radicals may be substituted by at least one group which is independently selectable from linear or branched, saturated or unsaturated, aliphatic or alicyclic C₁-C₆-alkyl groups and/or halogen atoms; and —[—(CH₂)ₓ—O—]_{y}—CH₃ groups where x=2-5 and y=1-20; and
the radical R is selected from linear or branched, saturated or unsaturated, aliphatic or alicyclic alkyl groups having 1 to 20 carbon atoms, where up to 6 hydrogen radicals may be substituted by OH groups; heteroaryl-C₁-C₆-alkyl groups having 3 to 8 carbon atoms, preferably 4 to 8 carbon atoms, in the heteroaryl radical and at least one heteroatom in the heteroaryl radical that is selected from N, O and S, wherein the alkyl groups may be linear or branched, saturated or unsaturated, aliphatic or alicyclic and wherein one or more hydrogen radicals on the heteroaryl radical may be substituted by at least one group which is independently selectable from linear or branched, saturated or unsaturated, aliphatic or alicyclic C₁-C₆-alkyl groups and/or halogen atoms; aryl-C₁-C₆-alkyl groups having 6 to 20 carbon atoms in the aryl radical, wherein the alkyl groups may be linear or branched, saturated or unsaturated, aliphatic or alicyclic and wherein one or more hydrogen radicals on the aryl radical may be substituted by at least one group which is independently selectable from linear or branched, saturated or unsaturated, aliphatic or alicyclic C₁-C₆-alkyl groups and/or halogen atoms; and —[—(CH₂)ₓ—O—]_{y}—CH₃ groups where x=2-5 and y=1-20; and

II) a component B) which consists of at least one of the following components:

B1) a salt that is formed from one of the anions [PO₄]³⁻, [HPO₄]²⁻, [H₂PO₄]⁻, [SO₄]²⁻, [HSO₄]⁻, [NO₃]⁻, [NO₂]⁻ or Cl⁻ and one, two or three of the cations stated in A), wherein the number of cations corresponds to the absolute value of the charge number of the respective anion;

B2) a salt that is formed from one or more bis(trifluoromethylsulfonyl)imide anions

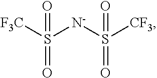 [SMILES: O=S(=O)([N-]S(=O)(=O)C(F)(F)F)C(F)(F)F]

and a lithium, potassium, cesium, magnesium, calcium, barium, nickel, cobalt, iron, scandium, lanthanum, zinc, gallium, cerium or aluminum cation, wherein the number of bis(trifluoromethylsulfonyl)imide anions corresponds to the absolute value of the charge number of the respective metal cation; and/or

B3) a zwitterionic compound that is formed from one of the cations stated in A), in which one of the radicals R, R¹, R² or R³ is a —(CH₂)ₓ—SO₃⁻ group where x=1-10.

In component B3), the radicals R, R¹, R² or R³ encompass all of those stated with respect to component A), where in addition each one of the radicals R, R¹, R² or R³ may also be a —(CH₂)ₓ—SO₃⁻ group where x=1-10 and one of the radicals R, R¹, R² or R³ also actually is a —(CH₂)ₓ—SO₃⁻ group where x=1-10.

In some embodiments, the mixture therefore has one or more of the components A) and one or more of the components B1, B2 and/or B3. Organic salts having an organic cation and the bis(trifluoromethylsulfonyl)imide anion are at any rate liquid and sufficiently stable under the reaction conditions present in the synthesis of methanol from carbon monoxide and hydrogen, carbon dioxide and hydrogen, or a mixture of carbon monoxide, carbon dioxide and hydrogen (temperatures in the range from 200° C. to 300° C. and pressures in the range from 50 bar to 300 bar).

However—due to the nature of the anion—these organic salts always have very hydrophobic, that is to say very low water- and/or methanol-solvent, properties. Due to the low solubility of these reaction products, there is insufficient achievement of the desired effect of a shift in the equilibrium by means of the dissolution of water and/or methanol into the ionic liquid during methanol synthesis. The (additional) use of one or more known water- and/or methanol-solvent organic ionic liquid(s) is out of the question, since, on account of the thermal instability thereof, this would lead to gaseous decomposition products that deactivate the catalyst used.

A mixture, however, of at least one of the above-listed components in A (e.g. a thermally stable, organic bis(trifluoromethylsulfonyl)imide salt) and at least one of the above-listed components B1 (salt that is formed from one of the anions [PO₄]³⁻, [HPO₄]²⁻, [H₂PO₄]⁻, [SO₄]²⁻, [HSO₄]⁻, [NO₃]⁻, [NO₂]⁻ or Cl⁻ and one, two or three of the cations stated in A), where the number of cations corresponds to the absolute value of the charge number of the respective anion), B2 (bis(trifluoromethylsulfonyl)imide salt having metallic cation) and/or B3 (zwitterionic compound) yields more highly water-solvent and methanol-solvent, thermally stable, low-melting sorption phases, which can successfully be used in methanol synthesis from carbon dioxide (CO₂) and/or carbon monoxide (CO) with hydrogen (H₂).

As experiments have shown, in the methanol synthesis using carbon dioxide and hydrogen, use of a mixture as liquid sorbent, by sorption of the reaction products water and methanol from the gaseous reaction phase, can for example achieve a shift in equilibrium by a factor of 4 or more, that is to say in the closed reactor more than four times more methanol is formed than would be possible under equilibrium conditions without the inventive mixture.

In some embodiments, the proportion by mass of component B), that is, the sum total of the proportions by mass of B1, B2 and/or B3, in the ionic liquid is in the range from 1% to 99%, e.g. in the range from 1% to 80%. A comparatively small proportion by mass of component B) is often already sufficient to achieve a markedly improved water- and/or methanol-solvent property of the mixture compared to component A) alone. By way of a few experiments, the person skilled in the art can determine that proportion by mass of the particular component B) which brings about, in the mixture with the particular component A), the necessary or desired dissolution properties with respect to water and/or methanol.

In some embodiments, the mixture is liquid at a temperature from 79° C., or from 49° C., or even from 19° C. Since the mixtures of the may be used as a liquid sorption phase, they may be liquid under the temperature conditions present during the sorption process.

If, by way of example, the sorption process takes place during the gas-phase methanol synthesis (“in-situ”), in which reaction temperatures in the range from approximately 100° C. to approximately 300° C. are present, a mixture must be chosen that is liquid in this temperature range. If, by way of example, the sorption process takes place outside of the reactor (“ex-situ”), where there may be lower temperatures here than in the reactor, or if the sorption process takes place in a region of the reactor in which lower temperatures prevail than in the “reaction zone” proper, a mixture must be chosen that is liquid at the lower temperatures present. The person skilled in the art can identify one or more respectively suitable mixtures by way of a few experiments.

In many cases the claimed mixtures have a melting point at any rate of below 80° C. if component A) is present in the mixture at a mass ratio of 20% or more. The respective melting point of a mixture can be ascertained by a simple experiment and the components of the mixture and their mixing ratios (ratios by mass) can be chosen such that both a desired or required melting point and a desired degree of methanol- and/or water-solvent property is achieved. In many cases, over a certain range of proportions by mass of component B), there is a linear relationship between the increase in methanol- and/or water-solvent property and the increase in the proportion by mass of component B).

The mixtures described herein are obtained by intensive mixing of the components said mixtures comprise. The individual components are all preparable by methods known to experts and are also commercially available (possibly as special order).

If the present application mentions that an alkyl, alkoxy and aminoalkyl group may be branched, this of course presupposes that the group has the required minimum number of 3 carbon atoms; if the present application mentions that a group is unsaturated, this of course presupposes that said group has at least 2 atoms that can in any case form a double bond with one another (for example at least 2 carbon atoms); if the present application mentions that a group is alicyclic, this of course presupposes that the group has at least 3, preferably 4, 5, 6 or more carbon atoms; if the present application mentions that in a group up to 6 hydrogen radicals may be substituted by OH groups, this of course presupposes that the group has the required number of substitutable hydrogen radicals (for a C₁ group at most 3 hydrogen radicals can be substituted; for a saturated C₂ group at most 5 hydrogen radicals; etc.). If mention is made in the present application of an aryl radical having 6 to 20 carbon atoms, this may be—depending on the number of carbon atoms—a monocyclic (for example with up to 10 carbon atoms) or polycyclic (for example starting from 10 carbon atoms) aryl radical, where the rings of a polycyclic aryl radical may be fused or maybe joined to one another by means of a C—C bond (such as in biphenyl). And, if mention is made in the present application of “halogen atoms”, this is to be understood to mean fluorine, chlorine, bromine and/or iodine atoms or radicals.

Some examples of the mixture are specified below: a) tributylmethylphosphonium bis(trifluoromethylsulfonyl)imide and lithium bis(trifluoromethylsulfonyl)imide;

b) methyltrioctylphosphonium bis(trifluoromethylsulfonyl)imide and lithium bis(trifluoromethylsulfonyl)imide;

c) trihexyltetradecylphosphonium bis(trifluoromethylsulfonyl)imide and lithium bis(trifluoromethylsulfonyl)imide;

d) tributylmethylphosphonium bis(trifluoromethylsulfonyl)imide and cesium bis(trifluoromethylsulfonyl)imide;

e) tributylmethylphosphonium bis(trifluoromethylsulfonyl)imide, lithium bis(trifluoromethylsulfonyl)imide and tributyl-4-sulfonyl-1-butanephosphonium;

f1) tributylmethylphosphonium bis(trifluoromethylsulfonyl)imide and tris(tetrabutylphosphonium) phosphate;

f2) tributylmethylphosphonium bis(trifluoromethylsulfonyl)imide, tris(tetrabutylphosphonium) phosphate and lithium bis(trifluoromethylsulfonyl)imide;

g1) tributylmethylphosphonium bis(trifluoromethylsulfonyl)imide and bis(tetrabutylphosphonium) sulfate;

g2) tributylmethylphosphonium bis(trifluoromethylsulfonyl)imide, bis(tetrabutylphosphonium) sulfate and lithium bis(trifluoromethylsulfonyl)imide;

h) tributylmethylphosphonium bis(trifluoromethylsulfonyl)imide, tetrakis(hydroxymethyl)phosphonium bis(trifluoromethylsulfonyl)imide and lithium bis(trifluoromethylsulfonyl)imide;

i) tributylmethylphosphonium bis(trifluoromethylsulfonyl)imide, tributylhydroxymethylphosphonium bis(trifluoromethylsulfonyl)imide and lithium bis(trifluoromethylsulfonyl)imide;

j) tributylmethylphosphonium bis(trifluoromethylsulfonyl)imide, tributyl-2,3-dihydroxypropylphosphonium bis(trifluoromethylsulfonyl)imide and lithium bis(trifluoromethylsulfonyl)imide.

For the identified binary mixtures, the mass ratio of the first component specified to the second component specified may be in the range from 4:1 to 7:3; for the identified ternary mixtures, the mass ratio of the first component specified to the second component specified to the third component specified is preferably in the range from 4:1:1 through 4:3:1 to 4:3:3. Mass ratios such as these are generally also suitable mass ratios for other binary and ternary mixtures of the present invention.

The teachings of the present disclosure are of course not limited to the examples given above and to the mass ratios specified. The present disclosure also describes the use of the mixtures in a methanol synthesis, especially a gas-phase methanol synthesis, as a liquid sorbent for methanol or methanol and water.

Some embodiments include methods for conducting a methanol synthesis, especially a gas-phase methanol synthesis, using carbon monoxide and hydrogen, carbon dioxide and hydrogen or a mixture of carbon monoxide, carbon dioxide and hydrogen as synthesis reactants in a reactor at a temperature in the range from 100° C. to 300° C. and a synthesis gas pressure in the range from 50 bar to 300 bar in the presence of a catalyst. As an example, some embodiments include methods comprising the following steps:
providing a liquid mixture as described above in the reactor;
converting carbon monoxide and hydrogen, carbon dioxide and hydrogen or a mixture of carbon monoxide, carbon dioxide and hydrogen to the synthesis product methanol or the synthesis products methanol and water in the reactor at a temperature in the range from 100° C. to 300° C. and a synthesis gas pressure in the range from 50 bar to 300 bar;
sorbing at least one subamount of at least one synthesis product, from the gas phase comprising at least one synthesis product, into the liquid mixture; and
continuously or discontinuously guiding the liquid mixture out of the reactor.

There are no particular limitations with respect to the reactor and it is possible to use any reactor that is known from the prior art for the specified purpose, for example including one as has been described in DE 10 2015 202 681 A1. In this regard and also with respect to the possible reaction regime, this document is also expressly incorporated by reference.

A suitable amount of the mixtures can be introduced into the reactor, wherein the reactor can subsequently be heated to a temperature in the range from 100° C. to 300° C. (e.g. in the region of 200° C.) that is suitable for the methanol synthesis or may already have a temperature suitable for this purpose upon introduction of the mixture. By these means, the mixture is in any event present in the reactor in liquid form. The mixture can of course also already have a temperature, outside of the reactor, at which the mixture is liquid, and the mixture can be introduced in liquid form into the reactor.

The pelletized or particulate catalyst required for the methanol synthesis can be arranged in the reactor above the mixture, for instance in a basket-like container. By introducing the synthesis gases carbon monoxide and hydrogen, carbon dioxide and hydrogen, or a mixture of carbon monoxide, carbon dioxide and hydrogen into the reactor and adjusting the synthesis gas pressure to a range from 50 bar to 300 bar (e.g. in the region of 80 bar) and a reactor temperature in the range from 100° C. to 300° C. (e.g. 200° C.), the synthesis gas reactants are converted to the synthesis gas product(s) methanol or methanol and water.

By way of the methanol-solvent or methanol- and water-solvent property of the liquid mixtures taught herein, at least one subamount of the synthesis product methanol or the synthesis products methanol and water pass over into the mixture, the result being that the equilibrium in the above reaction equations 1) and 2) is shifted in the direction of the reactants. Thus, a much larger proportion of reactants can be converted into the product(s) in the reactor than would be possible without the presence of the mixtures as taught herein. If a sufficient, desired or maximum possible amount of the reaction product(s) has passed over into the liquid mixture, this mixture is guided out of the reactor.

In some embodiments, during the process of the methanol synthesis or after the process of the methanol synthesis, the gas phase comprising at least one synthesis product can be introduced into the liquid mixture by means of a sparging stirrer. By these means, the sorption of the reaction product methanol or the reaction products methanol and water can be effected at a quickened rate compared to without such a measure. Of course, sorption of the synthesis product(s) from the gas phase can also be quickened by other suitable measures, such as by blowing the gas phase into the liquid mixture.

In some embodiments, the liquid mixture guided out of the reactor is depressurized and the synthesis product methanol—at least partially emerging from the mixture as a result—is recovered. The synthesis product methanol or the synthesis products methanol and water emerge(s) in gaseous form from a sufficiently hot/warm liquid mixture and can then, for example, be condensed by cooling.

In some embodiments, after the depressurization of the liquid mixture and the at least partial emergence from the mixture of the synthesis product methanol or the synthesis products methanol and water, the mixture is recycled back into the reactor. The recycling of the mixture may occur in a liquid state of the mixture. The recycling of the mixture into the reactor—as well as the guiding thereof out of the reactor—can be effected continuously or discontinuously. The same applies to the feeding of the synthesis reactants.

In some embodiments, a continuous methanol synthesis can be conducted, where at least subamounts of the synthesis product methanol or the synthesis products methanol and water can be transferred into the liquid mixture and continuously or discontinuously conducted out of the reactor. The mixture can be depressurized outside of the reactor, the emerging synthesis product(s) recovered and the mixture that is at least partially freed of the synthesis product(s) recycled into the reactor for renewed sorption of synthesis product(s).

In some embodiments, any suitable catalyst can be used. In some embodiments, the catalyst used is copper/zinc oxide on aluminum oxide, said catalyst being arranged in the gas phase of the reactor.

In some embodiments, the synthesis gas used is carbon dioxide and hydrogen; at least subamounts of the synthesis products methanol and water are sorbed into the liquid mixture in the reactor; the guidance of the mixture out of the reactor and the depressurization of the mixture (i.e. reducing the pressure over the mixture) is followed by the synthesis products methanol and water at least partially emerging from the mixture; at least the synthesis product methanol that has emerged is recovered (for example by condensation); and the depressurization of the liquid mixture and the at least partial emergence of the synthesis products from the mixture is followed by the mixture being recycled back into the reactor (in some cases, in liquid form).

In some embodiments, heat is extracted from the mixture prior to recycling into the reactor, that is to say in that the temperature of the mixture is lowered.

The mixtures described herein can be used in the liquid state as sorbent for methanol and/or water, specifically for all methods in which such sorption is desired, that is, not only within the context of a methanol gas-phase synthesis. In some embodiments, the mixtures are used “in situ”, that is to say in the methanol gas-phase synthesis, but the use is not restricted to such an application. Sorption of methanol and/or water can take place or be conducted under all suitable pressure (e.g. atmospheric pressure) and temperature conditions (e.g. starting from a temperature at which the particular mixture is liquid).

Where the term “sorbent” is used in the present application, this is to be understood in accordance with the generally recognized definition to mean an agent that is suitable for bringing about an accumulation of at least one substance (here: methanol and/or water) within a phase or on an interface between two phases (here: on the interface that is formed between a methanol- and/or water-containing gas phase and the liquid phase of the liquid mixture). A sorbent can thus bring about an accumulation of at least one substance within a phase (i.e. an absorption) and/or an accumulation on an interface (i.e. an adsorption), where for the mixtures of the present invention, when they are used as liquid sorbent, it can be assumed that exclusively or else wholly predominantly methanol and/or water is absorbed.

What is claimed is:

1. A mixture for use as liquid sorbent for methanol or methanol and water in methanol synthesis using carbon monoxide and hydrogen, carbon dioxide, and/or hydrogen as synthesis reactants, the mixture comprising:
I) a salt comprising a
bis(trifluoromethylsulfonyl)imide anion 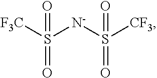 [SMILES: O=S(=O)([N-]S(=O)(=O)C(F)(F)F)C(F)(F)F]
and a cation selected from the group consisting of:
a quaternary ammonium cation of the general formula [NR¹R²R³R]⁺; or
a phosphonium cation of the general formula [PR¹R²R³R]⁺;
an imidazolium cation of the general formula 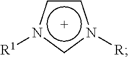
a pyridinium cation of the general formula 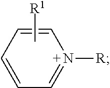
a pyrazolium cation of the general formula 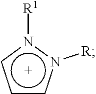
and
a triazolium cation of the general formula 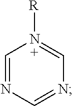
wherein, on the imidazole ring, pyridine ring, pyrazole ring or triazole ring, one or more hydrogen radicals is substituted by a group selected from the group consisting of: C₁-C₆-alkyl, C₁-C₆-alkoxy, C₁-C₆-aminoalkyl, C₅-C₁₂-aryl, and C₅-C₁₂-aryl-C₁-C₆-alkyl groups ;
wherein the aryl radical includes 6 to 20 carbon atoms; and
each of the radicals R¹, R², R³ is independently selected from the group consisting of: hydrogen, aliphatic or alicyclic alkyl groups having 1 to 20 carbon atoms with up to 6 hydrogen radicals substituted by OH groups, heteroaryl groups and/or heteroaryl-C₁-C₆-alkyl groups having 3 to 8 carbon atoms in the heteroaryl radical and at least one heteroatom in the heteroaryl radical selected from N, O, and S;
wherein one or more hydrogen radicals on the heteroaryl radical is substituted by at least one group selected from the group consisting of: aliphatic or alicyclic C₁-C₆-alkyl groups and/or halogen atoms, aryl and/or aryl-C₁-C₆-alkyl groups having 6 to 20 carbon atoms in the aryl radical;
wherein on the aryl radical one or more hydrogen radicals is substituted by at least one group selected from the group consisting of: aliphatic or alicyclic C₁-C₆-alkyl groups and/or halogen atoms; and —[—(CH₂)ₓ—O—]_{y}—CH₃ groups where x=2-5 and y=1-20; and
the radical R is selected from the group consisting of: aliphatic or alicyclic alkyl groups having 1 to 20 carbon atoms with up to 6 hydrogen radicals substituted by OH groups, heteroaryl-C₁-C₆-alkyl groups having 3 to 8 carbon atoms in the heteroaryl radical and at least one heteroatom in the heteroaryl radical selected from N, O and S, with one or more hydrogen radicals on the heteroaryl radical substituted by a group selected from the group consisting of: aliphatic or alicyclic C₁-C₆-alkyl groups and/or halogen atoms, aryl-C₁-C₆-alkyl groups having 6 to 20 carbon atoms in the aryl radical with one or more hydrogen radicals on the aryl radical substituted by at least one group selected from the group consisting of: aliphatic or alicyclic C₁-C₆-alkyl groups and/or halogen atoms and —[—(CH₂)ₓ—O—]_{y}—CH₃ groups where x=2-5 and y=1-20; and
II) at least one component selected from the group consisting of:
B1) a salt of an anion selected from the group consisting of: [PO₄]³⁻, [HPO₄]²⁻, [H₂PO₄]⁻, [SO₄]²⁻, [HSO₄]⁻, [NO₃]⁻, [NO₂]⁻, and Cl⁻, and one, two, or three of the cations of salt), wherein the number of cations corresponds to an absolute value of a charge number of the respective anion;
B2) a salt comprising one or more
bis(trifluoromethylsulfonyl)imide anions 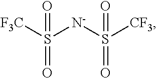 [SMILES: O=S(=O)([N-]S(=O)(=O)C(F)(F)F)C(F)(F)F]
and at least one component selected from the group consisting of: a lithium, potassium, cesium, magnesium, calcium, barium, nickel, cobalt, iron, scandium, lanthanum, zinc, gallium, cerium, and aluminum cations, wherein a number of bis(trifluoromethylsulfonyl)imide anions corresponds to an absolute value of a charge number of the respective metal cation; and
B3) a zwitterionic compound comprising a compound selected from the group of cations stated in A) wherein one of the radicals R, R¹, R² or R³ comprises a —(CH₂)ₓ—SO₃⁻ group where x=1-10.

2. The mixture as claimed in claim 1, wherein a proportion by mass of component B) in the mixture is in the range from 1% to 80%.

3. The mixture as claimed in claim 1, wherein the mixture has a liquid phase starting at a temperature above 19° C.

4. The mixture as claimed in claim 1, the mixture comprising:
a) tributylmethylphosphonium bis(trifluoromethylsulfonyl)imide and lithium bis(trifluoromethylsulfonyl)imide;
b) methyltrioctylphosphonium bis(trifluoromethylsulfonyl)imide and lithium bis(trifluoromethylsulfonyl)imide;
c) trihexyltetradecylphosphonium bis(trifluoromethylsulfonyl)imide and lithium bis(trifluoromethylsulfonyl)imide;
d) tributylmethylphosphonium bis(trifluoromethylsulfonyl)imide and cesium bis(trifluoromethylsulfonyl)imide;
e) tributylmethylphosphonium bis(trifluoromethylsulfonyl)imide, lithium bis(trifluoromethylsulfonyl)imide and tributyl-4-sulfonyl-1-butanephosphonium;
f1) tributylmethylphosphonium bis(trifluoromethylsulfonyl)imide and tris(tetrabutylphosphonium) phosphate;
f2) tributylmethylphosphonium bis(trifluoromethylsulfonyl)imide, tris(tetrabutylphosphonium) phosphate and lithium bis(trifluoromethylsulfonyl)imide;
g1) tributylmethylphosphonium bis(trifluoromethylsulfonyl)imide and bis(tetrabutylphosphonium) sulfate;
g2) tributylmethylphosphonium bis(trifluoromethylsulfonyl)imide, bis(tetrabutylphosphonium) sulfate and lithium bis(trifluoromethylsulfonyl)imide;
h) tributylmethylphosphonium bis(trifluoromethylsulfonyl)imide, tetrakis(hydroxymethyl)phosphonium bis(trifluoromethylsulfonyl)imide and lithium bis(trifluoromethylsulfonyl)imide;
i) tributylmethylphosphonium bis(trifluoromethylsulfonyl)imide, tributylhydroxymethylphosphonium bis(trifluoromethylsulfonyl)imide and lithium bis(trifluoromethylsulfonyl)imide; or
j) tributylmethylphosphonium bis(trifluoromethylsulfonyl)imide, tributyl-2,3-dihydroxypropylphosphonium bis(trifluoromethylsulfonyl)imide and lithium bis(trifluoromethylsulfonyl)imide.

5. The mixture as claimed in claim 4, the mixture comprising a binary mixture
wherein a mass ratio of the first component specified to the second component specified is in the range from 4:1 to 7:3.

6. A method for conducting a methanol synthesis using carbon monoxide and hydrogen, carbon dioxide, and/or hydrogen as synthesis reactants in the presence of a catalyst, the method comprising:
delivering a liquid mixture as claimed in claim 1 to the reactor;
converting carbon monoxide, carbon dioxide, and/or hydrogen to the synthesis product methanol in the reactor at a temperature in the range from 100° C. to 300° C. and a synthesis gas pressure in the range from 50 bar to 300 bar;
absorbing at least a portion of a synthesis product from a resulting gas phase comprising the synthesis product into the liquid mixture; and
discharging the liquid mixture out of the reactor.

7. The method as claimed in claim 5, further comprising introducing the gas phase into the liquid mixture with a sparging stirrer.

8. The method as claimed in claim 5, further comprising:
depressurizing the liquid mixture after removal from the reactor; and
recovering at least the methanol from the depressurized liquid mixture.

9. The method as claimed in claim 7, further comprising recycling the liquid mixture into the reactor after depressurization of the liquid mixture and recovery of the methanol.

10. The method as claimed in claim 5, wherein the catalyst comprises copper/zinc oxide on aluminum oxide arranged in the synthesis-gas gas phase of the reactor.

11. The method as claimed in claim 5, wherein:
the synthesis gas comprises carbon dioxide and hydrogen;
at least a portion of the synthesis products methanol and water are absorbed into the liquid mixture in the reactor;
discharging the mixture out of the reactor and depressurization of the mixture is followed by the synthesis products methanol and water at least partially emerging from the mixture;
and the depressurization of the liquid mixture and the at least partial emergence of the synthesis products from the liquid mixture is followed by recycling the liquid mixture into the reactor.

12. The method as claimed in claim 8, further comprising extracting heat from the liquid mixture prior to recycling the liquid mixture into the reactor.

13. The mixture as claimed in claim 4, the mixture comprising a ternary mixture wherein a mass ratio of the first component specified to the second component specified to the third component specified is in the range from 4:1:1 through 4:3:1 to 4:3:3.
